(19) United States
(12) Patent Application Publication
(10) Pub. No.: US 2009/0130190 A1
(43) Pub. Date: May 21, 2009

(54) Transdermal System for the Delivery of Sufentanil and Its Analogs

(75) Inventors: Armin Breitenbach, Leverkusen (DE); Peter Klaffenbach, Erkrath (DE); Ingo Lehrke, Koln (DE); Ulrike Vollmer, Dormagen (DE)

Correspondence Address: PATENT CORRESPONDENCE;ARNALL GOLDEN GREGORY LLP, 171 17TH STREET NW, SUITE 2100, ATLANTA, GA, 30363, US

(73) Assignee: LABTEC GMBH, Langenfeld (DE)

(21) Appl. No.: 12/255,466

(22) Filed: Oct. 21, 2008

Related U.S. Application Data
(63) Continuation-in-part of application No. PCT/EP2007/003498, filed on Apr. 20, 2007.

(30) Foreign Application Priority Data
Apr. 21, 2006 (DE) 102006019293.1

(51) Int. Cl. A61K 31/4436; A61K 9/70

(52) U.S. Cl. 424/449; 514/336

(57) ABSTRACT

Methods and systems for the transdermal delivery of sufentanil and its analogs are described, from patches having a unique pharmacodynamic profile that can be used to treat persistent pain over extended periods and acute pain episodes of limited duration.

### RELATION TO PRIOR APPLICATIONS

The present application claims priority to German patent application no. DE 10 2006 019 293.1, filed Apr. 21, 2006, U.S. provisional application No. 60/903,505, filed Feb. 26, 2007, and is a continuation in part of PCT/EP2007/003498, filed on Apr. 20, 2007.

### FIELD OF THE INVENTION

The present invention relates to methods and systems for the transdermal delivery of sufentanil and related analogs such as fentanyl. The invention also relates to a sufentanil patch having a unique pharmacokinetic profile that can be used to treat persistent pain over extended periods and acute pain episodes of limited duration.

### BACKGROUND OF THE INVENTION

Sufentanil is a powerful synthetic opioid in the fentanyl family of compounds that has proven utility in human medicine. The drug is chemically known as N-[4-(methoxymethyl)-1-[2-(2-thienyl)ethyl]-4-piperidinyl]-N-phenyl-propanamide, and is characterized by the following chemical structure:

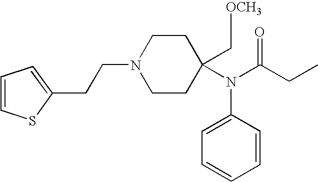

The drug has a number of analogs in the fentanyl family of compounds, including fentanyl, lofentanil, alfentanil, carfentanil, and remifentanil. Injectable formulations of sufentanil as its citrate salt have been approved in the United States for general anesthesia under the trademark Sufenta®.

The relative potencies of fentanyl and sufentanil have been reported in the literature, but inconsistently. For example, U.S. Pat. No. 4,588,580 reports that sufentanil is about 15 times more potent than fentanyl, while WO 2006/047362 discloses the sufentanil is from about 7.5 to 15 times more potent than fentanyl.

A transdermal patch is a drug containing adhesive bandage which, when applied to the skin, delivers the drug through the skin of a patient at a predetermined rate. The simplest type of patch is an adhesive monolith comprising a drug-containing matrix disposed on a flexible backing. The matrix is typically formed from a pressure sensitive adhesive so that the matrix can be adhered directly to the skin, although the matrix can also be formed from a non-adhesive material, in which case an additional adhesive layer is formed on the skin-contacting surface of the matrix. The rate at which the drug is delivered from these patches can vary due to differences in skin permeability among people and skin application sites. More complex patches are multilaminate or liquid reservoir types of patches in which a drug release-rate controlling membrane is disposed between the reservoir layer and the skin-contacting adhesive. This membrane, by decreasing the in vitro release rate of drug from the patch, can reduce the effects of variations in skin permeability.

The delivery of opioids through transdermal patches holds substantial promise in the treatment of chronic persistent pain because these patches can deliver pain medication constantly over the course of several days without the need for redosing. Numerous patents describe ways of transdermally administering fentanyl and its analogs. See, e.g., U.S. Pat. Nos. 4,466,953; 4,470,962; 4,588,580; 4,626,539; 5,006,342; 5,186,939; 5,310,559; 5,474,783; 5,656,286; 5,762,952; 5,948,433; 5,985,317; 5,958,446; 5,993,849; 6,024,976; 6,063,399 and 6,139,866. U.S. Pat. No. 4,588,580 also discloses that sufentanil has a solubility in skin of about 25-50% of fentanyl. However, narrow therapeutic indices associated with opioids, coupled with patient-to-patient variations in their response to opioids, dictates extreme caution in their administration.

The transdermal patch delivery of fentanyl in the United States became a commercial reality in 1990 with the regulatory approval of Duragesic® brand fentanyl patches by Janssen Pharmaceutica. The product is approved in several patch sizes that deliver area proportionate amounts of fentanyl through the skin to the systemic circulation, and has proven tremendously popular among patients who require constant plasma levels of this powerful analgesic. Duragesic® is applied to a patient for 3 days and is indicated for the treatment of chronic persistent pain. The Duragesic® fentanyl patch is intended to be removed and replaced with a fresh patch every 3 days, and it is contemplated that doses may be increased over time and that concurrent use of other analgesics may occur to deal with breakthrough pain. The patch suffers from a number of drawbacks, including a high lipophilicity, which results in accumulation of drug in the fatty tissue, and drug release from the fatty tissues back into the circulation in later times; a phenomenon known as the skin depot effect. In the clinic, this is significant because several applications of the patch must occur before titrating the dose upward, to ensure that maximum steady state blood concentrations have been reached before adjusting the dose.

Before now it has been thought that transdermal opioid patches designed for periodic replacement should deliver slow and steady amounts of drug over the course of a single application of the patch, and that this slow and steady delivery over a single application was needed to ensure clinically meaningful constancy in plasma concentrations over repeated patch applications. This thinking is most evident in WO 2006/047362 by Durect Corporation, which shows slow uptake of sufentanil over the first twenty-four hours of patch application, and fairly constant plasma levels of sufentanil over the next six consecutive days. According to the Durect application, the patch achieves substantially zero order kinetics over the course of up to seven days from application of a single patch. The examples describe a patch that contains 15.4 wt. % of a high MW polyisobutylene (Oppanol B100), 22 wt % of a low MW polyisobutylene (Oppanol B12), 48.5 wt. % polybutene, 6.5 wt. % CAB-O-Sil, and 7.7 wt. % sufentanil. Plasma levels from the Durect patch, as reported in WO 2006/047362, are plotted in FIGS. 6 and 7 hereto.

A prior art sufentanil patch is also described in WO 02/074286 by Alza Corporation, which describes a sufentanil patch that exhibits proportionate amounts of sufentanil penetration in vitro when the concentration of sufentanil in the patch is increased, and fairly steady sufentanil flux rates over the first 36 or 72 hours of patch administration, especially at lower doses. The examples describe a 2.54 cm² patch containing 0.25, 0.5, 0.75, 1.0 or 1.1 mg. sufentanil (corresponding to 2, 4, 6, 8 and 9 wt. % sufentanil), in a polyacrylate matrix, optionally with a permeation enhancer. FIG. 4 from Alza's application reports in vitro skin flux rates from the patch at the various sufentanil concentrations.

According to the Alza publication, the sufentanil patch described therein can achieve a standardized flux rate of from about 0.1 to about 10 mcg/(cm²·hr), and a normalized Cₘₐₓ (defined as Cₘₐₓ divided by nominal in vivo flux rate), of from about 0.04 to about 10 ng/(ml·(mg/hr)). The publication does not disclose how fast a sufentanil patch would reach Cₘₐₓ, but discloses a fentanyl patch that reaches Cₘₐₓ within about 24 hours.

A significant concern with opioid patches is patch diversion, and the illicit use of opioid remnant after the patch is removed and discarded. The prior art focus on constant delivery rates has hindered efforts to minimize this remnant because of the relationship between flux rates and drug concentrations in the patch. High drug concentrations typically must be present in the patch throughout the course of patch administration to prevent significant changes in drug flux. See WO 2006/047362 (reporting sufentanil concentrations above saturation). What is needed is a patch that provides clinically meaningful constancy in sufentanil plasma concentrations over repeated applications of the patch, and clinically significant pain control, without leaving high amounts of sufentanil residue in the patch after it is removed.

### SUMMARY OF THE INVENTION

Contrary to the teachings of the prior art, the present inventors have developed a patch that delivers sufentanil and its analogs for achieving clinically significant sustained pain relief—over a period of at least three days from a single patch—by achieving a high Cₘₐₓ in a relatively short time frame, relative to the patch's average delivery rate. In particular, the patch can reach plasma concentrations of at least 80% of Cₘₐₓ (maximum plasma concentration) within about twelve hours of patch application, and still provide sustained pain relief for periods of at least three days. By rapidly releasing a significant portion of the active ingredient in the patch in a short time frame, these patches provide long-term and short term analgesic effect while (1) lessening the amount of active ingredient required in the patch, and (2) reducing remnant drug in the patch when it is removed and discarded.

This rapid and efficient release of active ingredient into the bloodstream can be characterized by several kinetic variables. In one embodiment, these patches are characterized by a three day administration period, and a normalized Cₘₐₓ (defined as Cₘₐₓ divided by drug flux) over the three day period of greater than 15 or even 20 ng/(ml·(mg/hr)). In another embodiment, the patches are characterized by a standardized Cₘₐₓ (defined as Cₘₐₓ divided by the patch's surface area) of greater than 0.01 or even 0.03 ng/(ml·cm²). In still another embodiment, the patches are characterized by a Tₘₐₓ of about 18 hours or less, or an 80% Tₘₐₓ (i.e. time required to reach 80% Cₘₐₓ) of about 12 hours or less.

Another feature of the present invention, which did not become apparent until multiple dose studies were undertaken and steady state pharmacokinetic properties were analyzed, is the relationship between mean steady state plasma concentration and steady state nominal flux. Whereas theoretical calculations using intravenous infusion studies would predict that 1 mcg/hr would yield sufentanil plasma concentrations of less than 10 pg/ml (see example 9), and single dose bioavailability testing is consistent with these predictions, the inventors have discovered that at steady state the patches achieve greater than 15 pg/ml average plasma concentrations. Therefore, in another embodiment, the patches are characterized by a ratio of average plasma concentration to nominal flux of greater than 1.5*10⁻⁵ hr/ml.

Alternatively, the rapid delivery of the active ingredient from the current patches can be characterized by one or more of the following pharmacokinetic parameters (preferably defined over a three day application period):
a high Cₘₐₓ relative to the amount of active ingredient in the patch, and the amount of active ingredient that is ultimately delivered to the patient over the prescribed application period;
a large steady state average plasma concentration for the prescribed application period relative to the active ingredient loading in the patch;
a ratio of plasma concentrations Cₘᵢₙ to Cₘₐₓ of greater than about 1.5, wherein Cₘᵢₙ is identified after Cₘₐₓ has been reached; and/or
a low coefficient of variation in plasma concentrations, especially as compared to the commercial Duragesic® product.

The patches will typically be applied to patients who are already receiving opioid therapy, as a replacement for existing intravenous or oral opioid medications. Thus, for example, a patient receiving anywhere from about 60 to about 134 mg/day of oral morphine would initially be prescribed a patch that delivered about 3.5 mcg/hr of sufentanil (averaged over a prescribed administration period). Should the patient self-administer supplementary opioids for additional pain control during a patch application period, the patch dose can be increased based on the daily dose of supplementary opioids, using the ratio of 45 mg/day of oral morphine to a 1.75 mcg/hr increase in sufentanil delivery.

Still further embodiments relate to particular formulations used to make patches having these surprising pharmacodynamics. Thus, in one embodiment the invention relates to the use of polyisobutylene as the principle matrix component of a patch containing sufentanil or one of its analogs, in which the matrix comprises greater than 50 wt. % polyisobutylene, in which the ratio of low MW polyisobutylene to high MW polyisobutylene is preferably greater than 2:1, 3:1 or 4:1, and preferably less than 20:1. In another embodiment, the invention relates to the use of an irritation reducing agent such as calcium glycerophosphate in the formulation.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

FIG. 1 is a top plan view of an adhesive matrix patch constructed according to the present invention.

FIG. 2 is a side perspective view of an adhesive matrix patch constructed according to the present invention, showing a preferred three layer construction.

FIG. 3 is a graphical depiction of in vivo plasma levels over time for a 2.5 cm² patch constructed according to the present invention containing 0.625 mg of sufentanil base, in a phase I single dose bioavailability study in healthy volunteers (n=6) over seven days.

FIG. 4 is a graphical comparison of in vivo plasma levels over time, normalized based on 100% of Cₘₐₓ for each patch, for two different patches: (1) a 2.5 cm² patch constructed according to the present invention containing 0.625 mg of sufentanil base, and (2) a Duragesic® fentanyl patch having a nominal flux rate of 25 mcg/h. The sufentanil patch shows a Tₘₐₓ at 12 hours; the fentanyl patch at 24 hours.

FIG. 5 is a graphical comparison of the coefficients of variation (CV) in Cₘₐₓ for the two patches depicted in FIG. 4. The sufentanil patch is represented by the lower line; the fentanyl patch is depicted by the upper line.

FIG. 6 is a graphical comparison of in vivo plasma levels over time for three different patches: (1) a 2.5 cm² patch constructed according to the present invention containing 0.625 mg of sufentanil base, (2) a 2.0 cm² patch constructed according to WO 2006/047362 containing 0.91 mg of sufentanil base, and (3) a 2.0 cm² patch constructed according to WO 2006/047362 containing 1.7 mg of sufentanil base.

FIG. 7 is a graphical comparison of in vivo plasma levels over time for the same three patches depicted in FIG. 6, in which the plasma levels are normalized based on the application area of the patch.

FIG. 8 is a graphical depiction of in vivo plasma levels over time for a 2.5 cm² patch constructed according to the present invention containing 0.625 mg of sufentanil base, in a phase I multi-dose bioavailability study in healthy volunteers (n=7), wherein a fresh patch is applied every three days, depicting the third administration period.

FIG. 9 is a graphical depiction of the coefficient of variation for in vivo plasma levels over time for a 2.5 cm² patch constructed according to the present invention containing 0.625 mg of sufentanil base, in the phase I multi-dose bioavailability study depicted in FIG. 8, wherein a fresh patch is applied every three days.

FIG. 10 is a photographic comparison of sufentanil crystallization in (A) a patch without calcium glycerophosphate and (B) a patch with calcium glycerophosphate.

FIG. 11 is a photographic comparison of the matrix (left) and the open combs (right) of a sufentanil patch with a backing comprising open combs.

FIG. 12 is a photographic representation of a backing comprising open combs.

### DETAILED DESCRIPTION OF THE INVENTION

The present invention may be understood more readily by reference to the following detailed description of preferred embodiments of the invention and the Examples included therein.

### Definitions and Use of Terms

As used in this specification and in the claims which follow, the singular forms “a,” “an” and “the” include plural referents unless the context clearly dictates otherwise. Thus, for example, reference to “an ingredient” includes mixtures of ingredients, reference to “an active pharmaceutical agent” includes more than one active pharmaceutical agent, and the like.

Unless specified otherwise, the term “wt. %” as used herein with reference to the final product (i.e., the patch, as opposed to the formulation used to create it), denotes the percentage of the total dry weight contributed by the subject ingredient. This theoretical value can differ from the experimental value, because in practice, the patch typically retains some of the solvent used in preparation.

As used herein, the term “drug” refers to sufentanil and its analogs and includes sufentanil, fentanyl, lofentanil, alfentanil, carfentanil, remifentanil, and the like, and pharmaceutically acceptable salts and esters thereof. A preferred drug is sufentanil, and it is preferably used as the base molecule.

As used herein, the term “subsaturated patch” refers to a patch wherein the concentration of the drug is below its solubility limit. The matrix layer typically comprises a single phase polymeric composition wherein the drug and all other components are present at concentrations no greater than, and preferably less than, their saturation concentrations in the matrix.

As used herein, the term “single phase polymeric composition” refers to a composition in which the drug is solubilized in a polymer and is present at a concentration no greater than, and preferably less than, its saturation concentration in the matrix; wherein the active ingredient in combination with the polymer forms a single phase.

The term “first sufentanil patch system” refers to the first sufentanil patch system to be analyzed, and does not refer to the first sufentanil patch system ever to be administered to an individual during a sufentanil treatment regime. Thus, for example, a “first sufentanil patch system” can refer to the first, second, third etc. sequential system applied to an individual. When this document intends to reference the very first sufentanil patch system applied to an individual, it will be referred to as the “initial” patch system or the “first in sequence” patch system. In like manner, the “first” period does not necessarily refer to the first ever period. Rather, the term “initial” or other wording of like import will be used to refer to the first period in time.

### Discussion

As discussed above, the present invention provides a method and patch for the transdermal delivery of sufentanil and its analogs, preferably for the treatment of pain and the provision of sustained analgesia. The patch preferably delivers the drug at a rate and in an amount sufficient to induce and maintain analgesia over a period equaling or greater than two, three or four days, and up to 7 days, to a patient in need thereof. In one embodiment the pain is acute. In another embodiment the pain is chronic. In still another embodiment the pain is persistent moderate to severe chronic pain.

The patch typically comprises a protective flexible cover, an intermediate active ingredient layer having an adhesive surface opposite said protective cover, and a removable cover layer adjacent said adhesive surface. On application to the skin, active ingredient diffuses into and through the skin where it is absorbed into the bloodstream to produce a systemic analgesic effect. The onset of analgesia depends on various factors such as the solubility and diffusivity of the drug in the skin, thickness of the skin, concentration of the drug within the skin application site, concentration of the drug in the matrix layer, and the like. It is preferable that a patient experience an adequate effect within eight hours of initial application. However, this is significant only on the initial application. On repeated sequential applications, the residual drug in the application site of the patch is absorbed by the body at approximately the same rate as the drug from the new patch is absorbed into the new application area. Thus the patient should not experience any interruption of analgesia.

When continuous analgesia is desired the depleted patch is removed and a fresh patch applied to a new location. For example, the patch or patch system would be sequentially removed and replaced with a fresh patch or patch system at the end of the administration period to provide sustained relief from pain. Because absorption of the drug from the fresh patch into the new application area usually occurs at substantially the same rate as absorption by the body of the residual drug within the previous application site of the patch, blood levels will remain substantially constant. Additionally, it is contemplated that doses may be increased over time and that concurrent use of other analgesics may occur to deal with breakthrough pain.

The term “patch system” is used herein to refer to one or a plurality of patches applied during an administration period. A “patch system” could be made from several base patches dosed simultaneously, or it could be a single larger patch having a surface area and drug delivery rate equal to a multiple of the surface area and drug delivery rate of a base patch. In other words, the total surface area of the base patch could be defined as the base surface area and, because the delivery rate from transdermal patches is linearly related to the total surface area of the patch, successively larger patch systems would have surface areas of n·(base surface area), wherein n is an integer of from two to about ten. The base patch system of the present invention is preferably designed to achieve mean steady state plasma concentrations greater than or equal to the minimum effective plasma concentration, or to ensure that the plasma concentration at steady state does not fall beneath the lowest effective level.

A base patch will typically have a base delivery rate of from about 2.0 to about 7.0 mcg/hr, or any rate in between. In various embodiments, the base patch will have a base delivery rate of from about 2.5 to about 6.0 mcg/hr, from about 4.0 to about 5.0 mcg/hr, or from about 3.0 to about 3.5 mcg/hr. Alternatively, the patch can be described as having a base delivery rate of about 2.0±0.3, 2.5±0.3, 3.0±0.3, 3.5±0.3, 4.0±0.3, 4.5±0.3, 5.0±0.3, or 5.5±0.3, mcg/hr. In another alternative embodiment, the patch is described as having a base delivery rate of about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, or 5.5 mcg/h, optionally ±0.1, 0.2 or 0.3 mcg/hr. Successively larger patch systems will have a delivery rate equal to n·(base delivery rate), wherein n is an integer of from two to about ten.

### Patch Structure

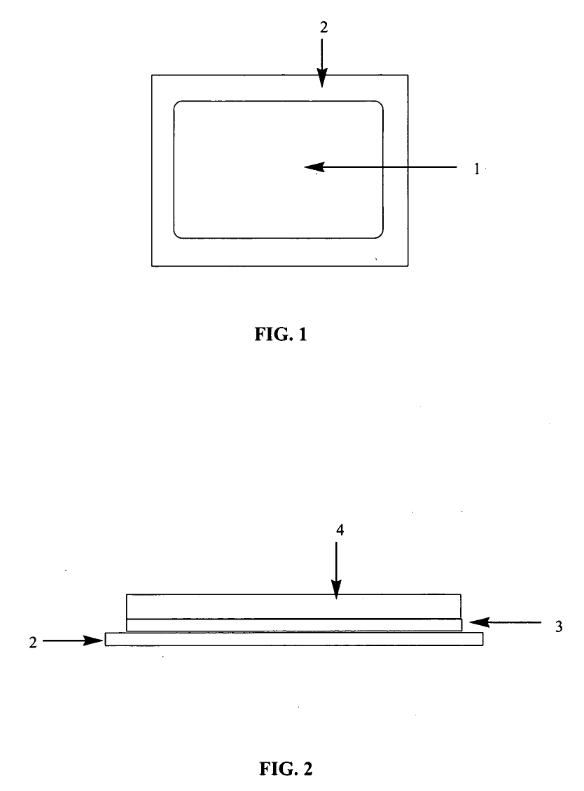

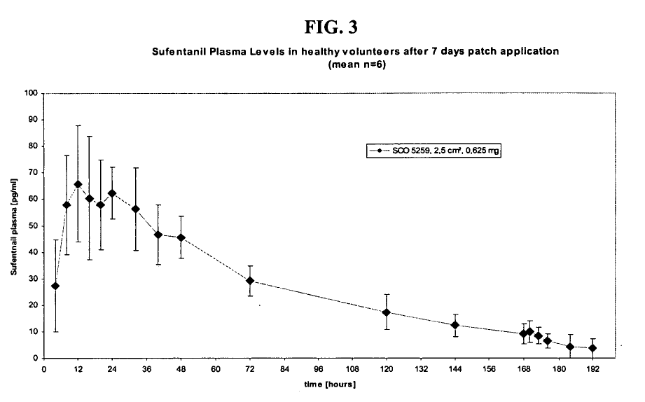

While the structure of patches of the present invention can vary, a preferred patch structure is depicted in FIGS. 1 and 2. As shown in FIGS. 1 and 2, the patch structure comprises the patch 1 that is applied to the skin, and a removable protective layer/release liner 2 that is removed from the patch before application. The patch 1 preferably comprises an adhesive layer or matrix 3 and a non-reactive cover layer 4. The active ingredient is integrated within the adhesive layer or matrix, and removal of the release liner 2 exposes the adhesive layer 3 which can then be applied to the skin. The patch structure including the patch 1 and removable protective layer 2 is preferably packaged in a foil package that is resistant to light and moisture. Other structures that the patches can assume include those with additional layers, such as an adhesive layer between the drug matrix and release liner, or a primer between the drug matrix and the cover layer, as taught by Park et al. in U.S. Pat. No. 6,190,690.

A base patch for the active ingredient (i.e., the structure applied to the skin) can be any shape, but is preferably rectangular in shape, with a surface area of from about 1.0 cm² to about 25 cm², most preferably about 1.5 cm² to about 5 or 10 cm². The thickness of the matrix layer preferably is such that from about 0.10 mg to about 2.0 mg., from about 0.15 to about 0.50 mg., or from about 0.20 to about 0.40 mg., are present in each square centimeter of the patch (most preferably about 0.25 mg/cm²). The thickness of the matrix layer can vary from about 5 to about 40 mil, and is preferably from about 10 to about 25 mil in thickness when applied wet during manufacture.

The non-reactive cover layer 4 plays an important part in the wearability of the patch. Because the transdermal system is applied to moving parts of the human body, a high degree of flexibility is necessary. It is also preferable that the cover layer 4 have good permeability to water vapor so as not to occlude the skin. Suitable materials for cover layer 4 include plastic films of polyethylene, vinyl acetate resins, ethylene/vinyl acetate copolymers, polyvinyl chloride, polyurethane, polypropylene, metal foils, woven fabrics, non-woven fabric, cloth and commercially available laminates. The backing material generally has a weight from about 2.0 to about 2.5 mg/cm². A bidirectional elastic material such as, for example, a non-woven polypropylene fabric, is particularly useful.

Protective layer 2 is preferably a sheet-like material constructed of materials that are inert to the matrix layer, and that can be readily separated from the matrix layer. Examples of the protective layer materials include polyurethane, polyvinyl acetate, polyvinylidene chloride, polypropylene, polycarbonate, polystyrene, polyethylene, polyethylene terephthalate, polybutylene terephthalate, paper, and the like, and combinations thereof.

### Pharmacokinetics

One of the most useful ways to characterize the patches of the current invention is by their pharmacokinetics. Unless otherwise stated, the pharmacokinetic parameters referenced herein can be used to describe the pharmacokinetics of an initial patch system or a patch system applied at steady state. It will further be understood that variability expressed as (±n) is optional.

As can be seen from the Figures, the patches of the current invention reach Cₘₐₓ in fairly short order, they reach a much higher Cₘₐₓ than a competitive patch, and they have average plasma concentrations larger than a competitive patch, even though the patches of the current invention have a lower load of sufentanil. The base 2.5 cm² patch containing 0.625 mg. of sufentanil, which could form the basis of other patches systems, produces opioid concentrations during the first seventy-two hours that are confined predominantly between (i.e. no more than 10, 20 or 40% outside of) a minimum effective level (30 pg/ml for sufentanil) and an average effective level (60 pg/ml for sufentanil), as described in the examples hereto.

The term “Cₘₐₓ” refers to the peak blood or plasma concentration of the drug when administered according to the methods of the present invention. The term Cₘₐₓ (80%) refers to a blood or plasma concentration of the drug that is reached before Cₘₐₓ is reached, amounting to 80% of the Cₘₐₓ for the drug. In one embodiment, the base sufentanil patch of the current invention can be described as having a Cₘₐₓ during the first seventy two hours of the initial dose of about 45, 50, 55, 60, 65, 70, or 75 pg/ml (±20%). At steady state, the base sufentanil patch of the current invention can be described as having a Cₘₐₓ of about 55, 60, 65, 70, 75, 80, 85 or 90 pg/ml (±20%). Larger patch systems would typically be designed to reach a multiple of the Cₘₐₓ achieved by the base sufentanil patch, i.e. n·(C_{max-base}), wherein n is an integer between 2 and 10. In another embodiment, the base sufentanil patch is described as having a Cₘₐₓ greater than about 45, 50, 55, or 60 pg/ml, and less than about 85, 80, 75 or 70 pg/ml, during the first seventy two hours of the initial dose, in any combination of mathematically possible endpoints. At steady state, the base sufentanil patch can be described as having a Cₘₐₓ greater than about 55, 60 or 65 pg/ml, and less than about 90, 85 or 80 pg/ml, in any combination of mathematically possible endpoints.

As used herein, the term “standardized Cₘₐₓ (pg/ml-cm²)” refers to the Cₘₐₓ (pg/ml) per unit area (cm²) of the active drug delivery area of the system, e.g., the area of the matrix layer. In one embodiment, the sufentanil patch is described as having a standardized Cₘₐₓ of 20.0, 25.0, 27.5, 30.0, 32.5, 35.0, or 40.0 pg/ml-cm² (+20%), during the initial dose or at steady state. In another embodiment, the sufentanil patch is described as having a standardized Cₘₐₓ greater than about 20.0, 25.0, 27.5, 30.0, 32.5, or 35 pg/ml-cm², and less than about 42.5, 40.0, 37.5 or 35.0 pg/ml-cm², in any combination of mathematically possible endpoints, during the initial dose or at steady state.

As used herein, the term “normalized Cₘₐₓ (pg/ml-(mcg/h))” refers to the Cₘₐₓ (pg/ml) divided by the average drug flux over a defined period of time (mcg/h). In one embodiment, the sufentanil patch is described as having a normalized Cₘₐₓ of 10.0, 11.0, 12.5, 14.0, 15.5, 17.0, 18.5, 20.0, 21.5, 23.0, 24.5 or 26.0 pg/ml-(mcg/h) (+5, 10 or 20%), when the patch is applied initially or at steady state. In another embodiment, the sufentanil patch is described as having a normalized Cₘₐₓ of greater than about 10.0, 11.0, 12.5, 14.0, 15.5, 17.0 or 18.5, 20.0 or 21.5 pg/ml-(mcg/h), and less than about 20, 25, 30 or 35 pg/ml-(mcg/h), when the patch is applied initially or at steady state, in any combination of mathematically possible endpoints.

In another principal embodiment, the invention is characterized based upon the average plasma concentrations observed for the patch at steady state, relative to the steady state flux of drug from the patch. Whereas infusion rates would suggest that 1 mcg/hr flux would result in a steady state plasma concentration of less than 10 pg/ml, the present inventors have discovered that the steady state delivery of about 3.5 mcg/hr by the patches of the present invention results in a steady state plasma concentration of about 53.8 pg/ml. I.e. the patches of the present invention result in a ratio of mean plasma concentration to nominal (i.e. steady state) flux of greater than 1.0*10⁻⁵ hr/ml, 1.2*10⁻⁵ hr/ml, 1.4*10⁻⁵ hr/ml, or even 1.5*10⁻⁵ hr/ml.

In still another embodiment, the patches are characterized by the time it takes to reach maximum plasma concentrations, or the time it takes to reach 80% of the maximum plasma concentration. In various embodiments, the patch or patch system is described as reaching Tₘₐₓ in about 24, 21, 18 or 15 hours or less, or an 80% Tₘₐₓ (i.e. time required to reach 80% Cₘₐₓ) of about 22, 18, 15, or 12 hours or less.

A particularly important characteristic of the patches of this invention is the quick onset of plasma concentrations, and the quick decrease in plasma concentrations when the patch is removed. In contrast to the commercial Duragesic patches, which exhibit a plasma half life of about 17 hours when removed from the skin, the patches of the present invention exhibit an average plasma half life of less than 12 or 10 hours, or ranging from 8 to 11, or 9 to 10 hours. At the front end, 50% Tₘₐₓ is preferably achieved in less than 10 or even 8 hours.

In another embodiment, the patches are characterized by a high Cₘₐₓ relative to the amount of active ingredient in the patch. Therefore, in one embodiment the patch is characterized by a loaded Cₘₐₓ (i.e. ratio of Cₘₐₓ to amount of sufentanil in the patch) (pg/ml·mg) of from about 50 to about 200 pg/ml·mg, from about 70 to about 150 pg/ml·mg, or from about 100 to about 135 pg/ml·mg. In another embodiment, the invention is characterized by a loaded Cₘₐₓ of 80, 90, 100, 110, 120, 130 or 140 pg/ml·mg (+20%).

In another embodiment, the patches are characterized by a high Cₘₐₓ relative to the amount of active ingredient that is ultimately delivered to the patient over the prescribed application period (“release load Cₘₐₓ”—defined as the ratio of Cₘₐₓ to amount of sufentanil released from the patch, as measured by the reduction in weight of the patch). Therefore, in one embodiment the patch is characterized by a release load Cₘₐₓ (pg/ml·mg) of from about 100 to about 400 pg/ml·mg, from about 140 to about 300 pg/ml·mg, or from about 200 to about 270 pg/ml·mg. In another embodiment, the invention is characterized by a release load Cₘₐₓ of 160, 180, 200, 220, 240, 260 or 280 pg/ml·mg (±20%).

In another embodiment, the patches are characterized by a large average plasma concentration for a prescribed application period relative to the active ingredient loading in the patch. In various embodiments, the patches achieve an average plasma concentration divided by the sufentanil load in the patch of greater than 4*10⁻⁸, 5*10⁻⁸, 6*10⁻⁸, 7*10⁻⁸, or 4*10⁻⁸ ml⁻¹

In still another embodiment, the patches can be characterized by the ratio of maximum to minimum plasma concentrations, over a prescribed administration period, wherein Cₘᵢₙ is identified after Cₘₐₓ has been reached. In various embodiment, the patches are characterized by a Cₘₐₓ:Cₘᵢₙ ratio of greater than about 1.5, 1.6, 1.7, 1.8, 1.9, or even 2.0, and less than about 3.0 or 2.5, over a forty-eight hour, seventy two hour, or ninety six hour administration period for a single patch system.

In another embodiment, the patches are characterized by a low coefficient of variation in maximum plasma concentrations, especially as compared to the commercial Duragesic® product. In various embodiment, the coefficient of variation for Cₘₐₓ is less than 40%, 30%, or even 25%.

A preferred embodiment of this invention is a patch that is bioequivalent to the patches described in the Examples of the present invention, when applied over a period of about two, three or four days. Thus, for example, in one embodiment the invention provides a patch that is bioequivalent to a reference patch, wherein said reference patch is a matrix patch made by a process comprising: (a) dissolving in hexane 1.0 weight parts of a high molecular weight polybutene, 5.0 weight parts of a low molecular weight polyisobutylene, and 2.0 weight parts polybutene to form an adhesive mixture; (b) dissolving 0.25 weight parts of sufentanil base in ethyl acetate to form a drug mixture; (c) mixing 0.25 weight parts of calcium glycerophosphate in said drug mixture to form a CGP mixture; (d) mixing said adhesive mixture and said CGP mixture to form a mixed liquid, and stirring said mixed liquid for one hour; (e) coating said mixed liquid onto a release liner at a sufentanil concentration of about 0.25 mg/cm²; (f) drying said coated liner; and (g) applying a backing foil to said dried coated liner.

In general, a standard bioequivalence study is conducted in a crossover fashion in a small number of volunteers, usually with 24 to 36 healthy normal adults. Single doses of the test and reference products are administered and blood or plasma levels of the drug are measured over time. Characteristics of these concentration-time curves, such as the area under the blood or plasma drug concentration-time curve (AUC), the peak blood or plasma concentration (Cₘₐₓ) of the drug, and/or time to peak plasma concentration (Tₘₐₓ), are examined by statistical procedures as described in greater detail hereinafter. In general, two one-sided statistical tests are carried out using the log-transformed parameter (AUC and Cₘₐₓ) from the bioequivalence study. The two one-sided tests are carried out at the 0.05 level of significance and the 90% confidence interval is computed. The test and the reference formulation/composition are considered bioequivalent if the confidence interval around the ratio of the mean (test/reference product) value for a pharmacokinetic parameter is no less than 80% on the lower end and no more than 125% on the upper end.

When comparing two different products whose drug administration rate is proportional to the size of the patch, the peak blood or plasma concentration of the drug (Cₘₐₓ) is standardized per unit area of the active drug delivery area of the system in order to establish bioequivalence. When comparing two different products having different drug administration rates per unit area, it is necessary to normalize the peak blood or plasma concentration of the drug (Cₘₐₓ) on the basis of the rate of drug administered to establish bioequivalence. Further detail regarding BE procedures can be found in FDA's July 1992 Guidance Document entitled “Statistical Procedures for Bioequivalence Studies Using a Standard Two-Treatment Crossover Design,” the contents of which are incorporated herein by reference.

### Dosing

In a particularly preferred embodiment the methods of treatment of the present invention are initiated in those patients who are opioid tolerant, and the patches are dosed based on the dose of opioid being administered daily to the patient (i.e. the opioid demand). Thus, for example, if a patient is currently taking about 90 mg/d of oral morphine, it would be advisable to start the patient with a dose of sufentanil of about 3.5 mcg/hr. From this point, the substitution of the sufentanil would essentially be a linear relationship. Therefore, in one embodiment the methods of the present invention are initiated in a patient that has an existing opioid demand equipotent to n·90 mg/d of oral morphine (i.e. the patient is receiving opioids of a type and in an amount that is equipotent to about 90 mg/day of oral morphine, or a multiple thereof), and said first patch system delivers n·(6.0±0.5), n·(6.5±0.5), n·(5.0±0.5), n·(4.5±0.5), n·(4.0±0.5), n·(3.5±0.5), n·(3.0±5) or n·(2.5±0.5) mcg/hr of sufentanil, wherein n is an integer of from 1 to 12. Alternatively, the patient that has an existing opioid demand equipotent to n·90 mg/d of oral morphine may be initially prescribed a patch system that delivers n·(1.0 to 8.5), n·(2.5 to 4.0), or n·(4.0 to 5.5) mcg/hr of sufentanil, wherein n is an integer of from 1 to 12.

Of course, a patient will not always be prescribed exactly 90 mg/d of oral morphine or a multiple thereof, in which case the conversion may be established using the following table, which is derived from the prescribing information for Duragesic®:

RECOMMENDED INITIAL SUFENTANIL PATCH DOSAGE
BASED UPON DAILY ORAL MORPHINE DOSE

| Oral 24-hour Morphine | Sufentanil Patch |
|---|---|
| (mg/day) | Dose (mcg/h) |
| 60-134 | 3.5 ± 0.5 |
| 135-224 | 7.0 ± 1.0 |
| 225-314 | 10.5 ± 1.5 |
| 315-404 | 14.0 ± 2.0 |
| 405-494 | 17.5 ± 2.5 |
| 495-584 | 21.0 ± 3.0 |
| 585-674 | 24.5 ± 3.5 |
| 675-764 | 28.0 ± 4.0 |
| 765-854 | 31.5 ± 4.5 |
| 855-944 | 35.0 ± 5.0 |
| 945-1034 | 38.5 ± 5.5 |
| 1035-1124 | 42.0 ± 6.0 |

It will be understood that many patients will be on existing treatments of opioids other than oral morphine when switched to the patch system, and the dose of the patch system can be derived from equipotency charts that show the relative doses of opioids required to give the same degree of analgesia. An accepted equipotency chart found in the prescribing information for Duragesic is provided below:

EQUIANALGESIC POTENCY CONVERSION

Equianalgesic
Dose (mg)

| Name | IM | PO |
|---|---|---|
| Morphine | 10 | 60 |
| Hydromorphone | 1.5 | 7.5 |
| Methadone | 10 | 20 |
| Ocycodone | 15 | 30 |
| Levorphanol | 2 | 4 |
| Oxymorphone | 1 | 10 (PR) |
| Meperidine | 75 | — |
| Codeine | 130 | 200 |

Thus, for example, oral oxycodone is twice as potent as oral morphine, and a patient receiving 45 mg/d of oral oxycodone would initially preferably be prescribed a patch system that delivers about 3.5 mcg/hr, which is the same dose that a patient on 90 mg/d of oral morphine would preferably receive.

In any of the foregoing methods of treatment, it will be understood that the first patch system will most often be only the first in a series of treatments for delivering sustained analgesia over extended periods of time. Therefore, when a second patch system is applied after the first period ends and the first patch system is removed, the invention further provides adhering a second patch system to the skin of said patient after said first period, for a second period of at least two or three days, wherein said second patch system demonstrates a second Cₘₐₓ, and wherein said first and second patch systems are defined by an identical composition and size. The first Cₘₐₓ and second Cₘₐₓ are preferably the same or similar, and in various embodiments the first and second Cₘₐₓ values vary by 20%, 15%, 10% or 5% or less. Alternatively, the first and second Cₘₐₓ values may vary by 5%, 10%, 15% or 20% or more.

In one embodiment the dose of active ingredient is adjusted upward after the first patch administration if the patient experiences inadequate pain control during the first period of application. In such an embodiment the second patch system will have an in vivo flux rate greater than said first patch system. The additional sufentanil dose can often be calculated based upon the amount of supplemental opioid taken by the patient during the first period, using the ratio of 1.75 mcg/hr sufentanil (±0.25 mcg/hr) for every 45 mg/d oral morphine taken by the patient during the first period, or equipotent opioid dose. Therefore, in another embodiment, the methods of the present invention are characterized by a patient who experiences inadequate pain control during the first period, further comprising: (a) administering a supplemental opioid dose equipotent to n·45 mg/d oral morphine during said first period, and (b) administering a second patch system having an in vivo flux rate equal to the in vivo flux rate of said first system, plus n·(1.75±0.25) mcg/hr of sufentanil, wherein n is an integer of from 1 to 5.

In another embodiment the dose of active ingredient is not adjusted upward until after the initial and second or subsequent patch administration if the patient experiences inadequate pain control during the initial and second periods of application. In such an embodiment the second patch system will have a size and a composition the same as the initial patch system. If the initial dose continues to be inadequate after the second dose, a third patch system may be applied having additional sufentanil. The additional sufentanil dose can often be calculated based upon the amount of supplemental opioid taken by the patient during the second period, using the ratio of 1.75 mcg/hr sufentanil (±0.25 mcg/hr) for every 45 mg/d oral morphine taken by the patient during the second period, or equipotent opioid dose. Therefore, in another embodiment, the methods of the present invention are characterized by a patient who experiences inadequate pain control during the initial period, further comprising: (a) administering a supplemental opioid dose equipotent to m·45 mg/d oral morphine during said second period, and (b) administering a third patch system having an in vivo flux rate equal to the in vivo flux rate of said first system, plus m·(1.75±0.25) mcg/hr of sufentanil, wherein m is an integer of from 1 to 5.

### Patch Composition

### Matrix

As discussed above, the matrix preferably comprises a single phase polymeric composition containing an amount of active ingredient sufficient to induce and maintain analgesia in a human for at least three days. In preferred embodiments, the matrix layer comprises about 0.05 to about 1.75 mg/cm² of sufentanil; preferably about 0.07 to about 1.50 mg/cm² of sufentanil; preferably about 0.08 to about 1.25 mg/cm² of sufentanil; more preferably about 0.09 to about 1.0 mg/cm² of sufentanil; more preferably about 0.1 to about 0.75 mg/cm² of sufentanil; more preferably about 0.12 to about 0.5 mg/cm² of sufentanil; and even more preferably about 0.2 to about 0.4 mg/cm² of sufentanil. The sufentanil is preferably in a base form and it is preferably completely dissolved.

A number of matrices for manufacturing patches are known in the art and are generally suitable for use in forming matrix layer 3. In preferred embodiments, the matrix layer 3 is formed from a pharmaceutically acceptable pressure sensitive adhesive such as, for example, polyacrylates, polysiloxanes, polyisobutylene (PIB), polyisoprene, polybutadiene, styrenic block polymers, and the like. Examples of styrenic block copolymer-based adhesives include, but are not limited to, styrene-isoprene-styrene block copolymer (SIS), styrene-butadiene-styrene copolymer (SBS), styrene-ethylenebutene-styrene copolymers (SEBS), and di-block analogs thereof. Acrylic polymers may be comprised of a copolymer or terpolymer comprising at least two or more exemplary components selected from the group comprising acrylic acids, alkyl acrylates, methacrylates, copolymerizable secondary monomers or monomers with functional groups. Examples of monomers include, but are not limited to, vinyl acetate, acrylic acid, methacrylic acid, methoxyethyl acrylate, ethyl acrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate, methoxyethyl methacrylate, and the like.

In one embodiment the matrix layer of the patches is characterized by a polyisobutylene content of greater than about 40 wt. %, 50 wt. %, 60 wt. %, or even 70 wt. %. In another embodiment the matrix layer is characterized by a polyisobutylene content of greater than 25 wt. %, 50 wt. %, 60 wt. %, or even 70 wt. %, wherein said polyisobutylene is a combination of high molecular weight and low molecular weight polyisobutylene chains, and the ratio of low MW polyisobutylene to high MW polyisobutylene is greater than 2:1, 3:1 or 4:1. For purposes of this invention, a high MW polyisobutylene is defined as a polyisobutylene having a molecular weight greater than 250,000, 650,000, or 1,000,000 g/mol, and a low MW polyisobutylene is defined as a polyisobutylene having a molecular weight of less than 250,000, 100,000 or 40,000 g/mol.

### Active Ingredient

The matrix layer also contains the active ingredient of the patch (i.e. sufentanil or an analog thereof) solubilized in the matrix. The drug can be in base, salt or ester form, though it is preferably supplied in the form of its base. The matrix layer preferably comprises from about 1 to about 20 wt. %, from about 1.5 to about 10 wt. %, or from about 2 to about 5 wt. %, based on the solids content of the matrix layer. In a particularly preferred embodiment, the patch has an area of about 2.5 cm², or a multiple thereof, and comprises about 0.25 mg of sufentanil per cm²), preferably at a weight percentage of about 2.9%. In a preferred embodiment, the patch is packaged so that the drug remains solubilized in this concentrated state, without recrystallization, for at least 6 months, one year, eighteen months or two years.

### Irritation Reducing Agents

In one embodiment, the patches of the present invention comprise one or more undissolved components, such as an irritation reducing agent. Surprisingly, it has been found that patches containing undissolved particles are better tolerated than patches free of undissolved particles. It is believed that the undissolved particles can act as nucleation/crystallization centers for the active ingredients. Because of the homogeneous distribution of the undissolved particles within the matrix, any crystals of the active ingredient that form in the matrix remain very small and evenly distributed throughout the matrix. In this way, the undissolved components can prevent the active ingredient from forming localized agglomerates of crystals that may cause irritation

In one embodiment, the undissolved particles are selected from pectins (i.e. natural 1,4-glycosidic high molecular weight carbohydrates), arabinans, galactans and analogs. In a preferred embodiment the undissolved component is a nonsaccharide polyhydric alcohol phosphate ester, or a mono- or divalent metal ion salt thereof, such as the calcium, sodium, potassium, ammonium or magnesium salt for thereof. A particularly preferred undissolved component is calcium glycerophosphate. The undissolved particles are preferable present in the matrix in an amount greater than about 0.5 wt. %, 1.0 wt. %, or 3.0 wt. %, and less than about 10 wt. %.

### Inactive Ingredients

In certain embodiments, a plasticizer or tackifying agent is incorporated in the adhesive composition to improve the adhesive characteristics. Examples of suitable tackifying agents include, but are not limited to, aliphatic hydrocarbons; aromatic hydrocarbons; hydrogenated esters; polyterpenes; hydrogenated wood resins; tackifying resins such as ESCOREZ, aliphatic hydrocarbon resins made from cationic polymerization of petrochemical feedstocks or the thermal polymerization and subsequent hydrogenation of petrochemical feedstocks, rosin ester tackifiers, and the like; mineral oil and combinations thereof. A particularly preferred tackifying agent is polybutene. In various embodiments, the matrix comprises polyisobutylene and polybutene at a weight ratio of from about 1:1 to about 6:1, or from about 2:1 to about 5:1, preferably about 3:1.

Penetration enhancers can optionally be employed in the patches of the present invention. Penetration enhancers are well known and are referred to in the art by terms such as skin-penetration enhancers, accelerants, adjuvants, and sorption promoters, all of which are referred to collectively herein as “penetration enhancers.” Agents within this class have diverse mechanisms of action, and include agents that improve the solubility and diffusibility of a drug within the multi-monomer polymeric matrix and those which improve percutaneous adsorption, for example, by changing the ability of the stratum corneum to retain moisture, softening the skin, improving the skin's permeability, acting as penetration assistants or hair-follicle openers or changing the state of the skin including the boundary layer.

Various pharmaceutically acceptable additives and excipients may also be incorporated into the matrix including tackifying agents, binders and rheological agents (i.e., thickeners). Other additives and excipients include diluents, stabilizers, fillers, clays, buffering agents, biocides, humectants, anti-irritants, antioxidants, preservatives, plasticizing agents, cross-linking agents, flavoring agents, colorants, pigments and the like. In a preferred embodiment the matrix also includes calcium glycerophosphate, which preferably constitutes the only undissolved component in the matrix.

The matrix compositions according to the present invention can be prepared by first mixing appropriate amounts of the matrix material in volatile polar and/or non-polar organic liquids. An appropriate amount of active ingredient is then added to the matrix material and the ingredients are thoroughly mixed. The active ingredient is preferably added as a solution dissolved in methanol, ethanol, 2-propanol or ethyl acetate. The mixture of the matrix composition is next formed into a film at ambient temperature, preferably by coating or casting at a controlled specified thickness onto a flexible sheet material, such as the protective layer 2, followed by evaporation of the volatile solvents at elevated temperatures (e.g., by passing through an oven). The matrix that has been coated or cast on the flexible sheet material is then laminated to another flexible sheet material, cover layer 4. Appropriate size and shape individual patches are then cut and packaged (e.g., pouched).

### Backing

In one embodiment, the patch comprises a backing with a comb structure. Surprisingly, it has been found that patches with a backing with a comb structure are better tolerated than patches with a backing without a comb structure. It is believed that the corners of the combed structure can act as nucleation/crystallization centers for the active ingredients. Crystallization at the interface of the comb structure of the backing and the matrix can prevent crystals from forming agglomerates in other parts of the matrix that may contact the skin and cause irritation. In this way, the open combs can prevent the active ingredient from forming crystals in locations that may cause skin irritation.

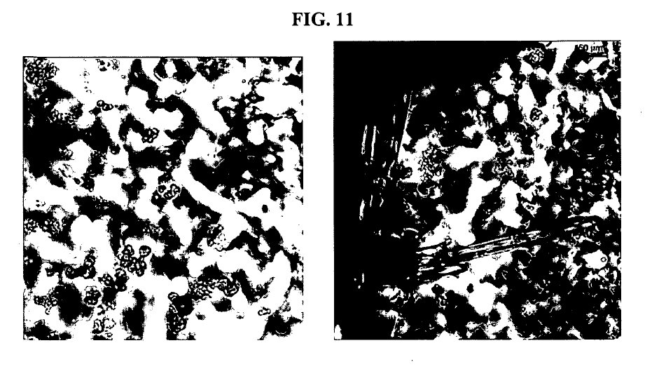

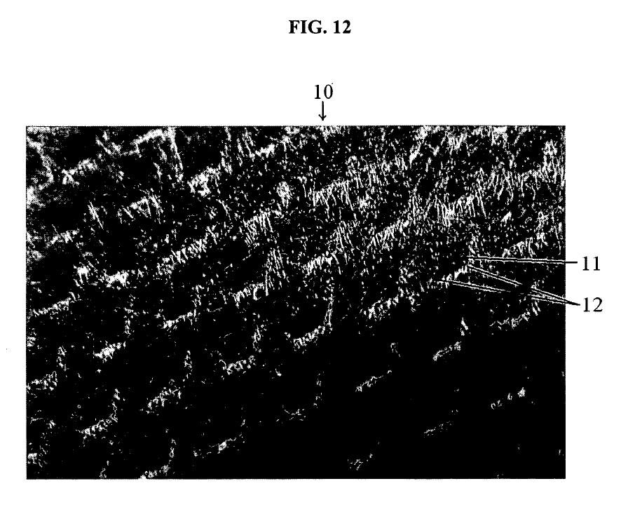

FIG. 11 shows a photograph of one embodiment of a suitable backing 10. The backing 10 can include a plurality of comb structures 11. The comb structures 11 can include any number of corners 12 that can act as nucleation/crystallization centers for the active ingredients.

### EXAMPLES

The following examples are put forth so as to provide those of ordinary skill in the art with a complete disclosure and description of how the compounds claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at room temperature, and pressure is at or near atmospheric.

### Example 1

### Determination of Minimum and Average Effective Plasma Levels for Sufentanil

Table A below summarizes the median sufentanil i.v. infusion rates, and resulting steady state plasma levels, from sufentanil infusion for intensive case applications and for sustained analgesia in post-surgical or chronic pain applications. In the intensive care unit (ICU), the goal is generally moderate patient sedation (somnolent but easily aroused) and effective analgesia. For sustained pain control, the goal is effective pain control with minimal to moderate sedation. In these studies, steady-state sufentanil infusions were generally combined with additional bolus injections of sufentanil as a pre-medication and as demanded by the patient through patient controlled analgesia (PCA) pumps.

The “average intravenous infusion rate for sustained analgesia,” from six studies in Table A (using the midpoint of the range from Coda et al. (1997)) was 0.104 mcg/kg/h. The average of the mean or median plasma levels from the three pain studies in Table A (the “average analgesic plasma level”) was found to be 63 pg/ml, using the midpoint value from the quoted range from Geller et al. (1993). The three analgesia studies used to derive this average were all postoperative pain studies in which intravenous sufentanil was used as the sole analgesic following major surgery. Pain was generally described as well controlled by sufentanil in these studies.

TABLE A

| Mean | | Mean | | |
|---|---|---|---|---|
| Intravenous | Mean Plasma | Duration of | | |
| Infusion Rate | Concentration | Infusion | | |
| (microg/kg/h) | (pg/ml) | (hours/days) | Type of Pain | Reference |
| 0.10 | 110 | 24 h (1 d) | Postoperative-major | (Menigaux et al. |
| | | | abdominal or urological | ′01) |
| | | | surgery | |
| 0.11 | ~30-60 | 24 h (1 d) | Postoperative-major | (Geller et al. ′93) |
| | | | abdominal surgery | |
| 0.12 | 35 | 17 h | Postoperative-elective | (Lehmann et al. ′91) |
| | | | major gynecological | |
| | | | surgery | |
| [0.15] | [~110] | [6 h] | [None-normal human | [(Fisher et al. ′03)] |
| | | | volunteers] | |
| 0.13 | | 24 h (1 d) | Postoperative- | (Cohen et al. ′88) |
| | | | cesarean section | |
| 0.08 | | 24 h (1 d) | Postoperative-misc. | (Ved et al. ′89) |
| | | | surgeries | |
| ~0.06-0.11 | | 312 h (13 d) | Oral mucositis pain | (Coda et al. ′97) |
| | | | following bone marrow | |
| | | | transplantations | |

One post-operative study attempted to determine the “minimum effective analgesic plasma level,” which was estimated at 30 pg/ml (Lehmann et al. '91). This minimum effective level is 48% of the average effective drug level estimated from the postoperative studies in Table A. Assuming that steady state plasma levels are linearly related to infusion rates at these lower dosing levels, the minimum effective plasma level should be achieved following a “minimum effective intravenous infusion rate” of 0.050 mcg/kg/h. These minimum and mean values are summarized in Table B below.

TABLE B

| Intravenous infusion rate | Plasma sufentanil concentration |
|---|---|
| Minimum effective rate-0.050 | Minimum effective level-30 pg/ml |
| microg/kg/h | |
| Average effective rate-0.104 | Average effective level-63 pg/ml |
| microg/kg/h | |

### Example 2

### Composition of Sufentanil Patch

Table C sets forth an exemplary composition for the patches of the present invention.

TABLE C

| Component | Function | mg per sqcm |
|---|---|---|
| Securon PP ® (Corovin) | Non-woven backing | 2.10 |
| Scotchpack 1022 ® (3M) | Release Liner | — |
| | (removed at application) | |
| Sufentanil Base | API | 0.25 |
| Calcium glycero phosphate | Skin care agent | 0.25 |
| Oppanol ™ B100 | Adhesive polymer | 1.00 |
| (high molecular PIB) | | |
| Oppanol ™ B10 | Adhesive polymer | 5.00 |
| (low molecular PIB) | | |
| Polybutene | Tackifier | 2.00 |
| Ethyl acetate | Solvent | Solvent, not part |
| | | of final product! |

Oppanol B100 is polyisobutylene with a molecular weight of > 1,000,000 g/mol.
Oppanol B10 is polyisobutylene with a molecular weight of 40,000 g/mol.

### Example 3

### Method of Making Sufentanil Patch

The required amount of the three excipients forming the PIB adhesive (Oppanol B100, Oppanol B10 and Parapol 920)—are weighed and dissolved in hexane under stirring. Sufentanil is dissolved in ethyl acetate. Calcium glycerol phosphate is added to the clear drug solution under stirring to yield a homogenous suspension. The adhesive solution is added slowly to the drug solution under stirring and stirred for an additional hour to yield a homogenous mixture without any air bubbles. In bench scale manufacturing this mixture is then treated with ultra-sound for 2 times 15 min (to remove bubbles if any).

The mixture is coated onto the release liner (the mixture has to be kept under constant stirring to avoid segregation of the dispersed calcium glycerol phosphate). The coated film is dried at room temperature for 10 min followed by 20 min at 750C. The backing foil is applied and the patches are punched out of the resulting laminate, followed by primary packaging in heat sealed individual pouches.

### Example 4

### Bioavailability Testing of Sufentanil Patch

A single center, one dose, open label study to investigate the safety, tolerability and pharmacokinetics of sufentanil transdermal patches was undertaken using a parallel groups design. FIG. 3 is a graphical depiction of in vivo plasma levels over time for a 2.5 cm² patch constructed according to the present invention containing 0.625 mg of sufentanil base, in a phase I single dose bioavailability study in healthy volunteers (n=6) over seven days. Other kinetic variables are reported below in Table E.

TABLE D
SUFENTANIL - KINETIC VARIABLE 0-192 H

| | AUC_{0-tz} | AUC_{0-∞} | Cₘₐₓ | tₘₐₓ | t_{1/2} | Coeff. of |
|---|---|---|---|---|---|---|
| Subj | [pg/ml · h] | [pg/ml · h] | [pg/ml] | [h] | [h] | Corr. |
| N | 6 | 6 | 6 | 6 | 6 | |
| Mean | 5231.3 | 5217.1 | 72.23 | 18.00 | 16.37 | |
| SD | 944.0 | 993.3 | 15.78 | 5.51 | 4.62 | |
| CV | 18.0 | 18.7 | 21.8 | 30.6 | 28.2 | |
| Min | 3910.4 | 3946.9 | 54.12 | 12.00 | 12.36 | −0.997 |
| Med | 5052.3 | 5112.2 | 71.51 | 18.01 | 14.61 | −0.948 |
| Max | 6503.9 | 6655.2 | 100.26 | 24.00 | 25.10 | −0.786 |
| GeoM | 5159.3 | 5238.9 | 70.89 | | 15.92 | |
| G_CV | 18.5 | 19.2 | 21.1 | | 25.6 | |

### Example 5

### Comparative Bioavailability Testing of Duragesic®

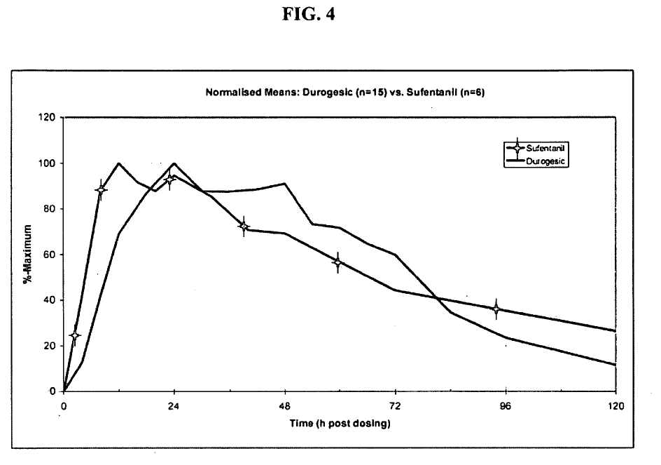

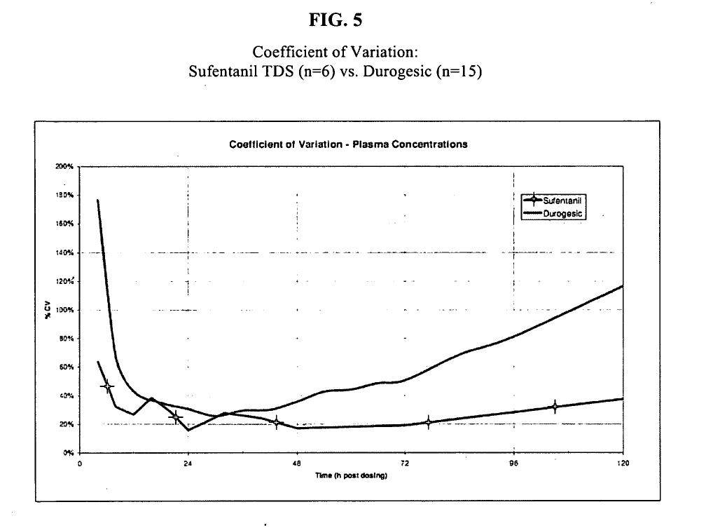

A separate bioavailability study was made of commercially available Duragesic patches, and the results graphically versus the pharmacokinetics plotted in FIG. 3. FIG. 4 is a graphical comparison of in vivo plasma levels over time, normalized based on 100% of Cₘₐₓ for each patch, for two different patches: (1) a 2.5 cm² patch constructed according to the present invention containing 0.625 mg of sufentanil base, and (2) a Duragesic fentanyl patch having a nominal flux rate of 25 mcg/h. FIG. 5 is a graphical comparison of the coefficients of variation (CV) in Cₘₐₓ for the two patches depicted in FIG. 4.

### Example 6

### Comparative Bioavailability Analysis of Durect Sufentanil Patch

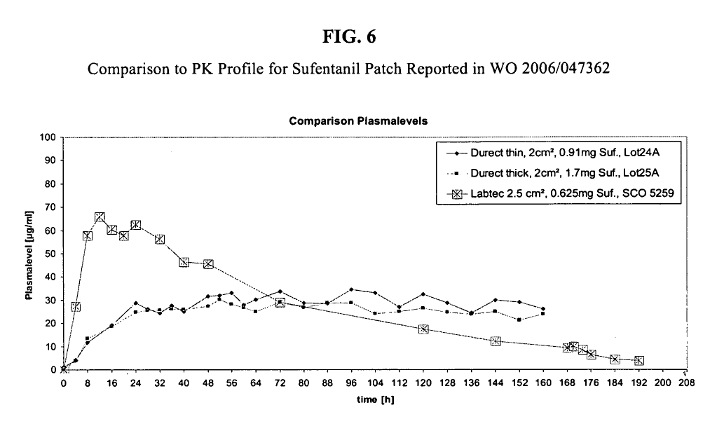

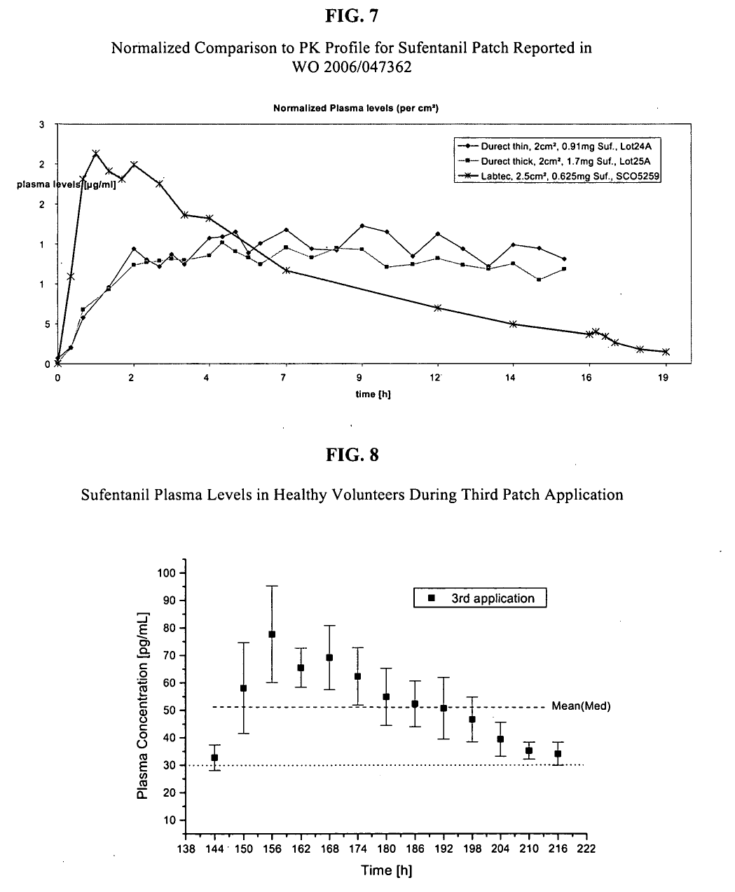

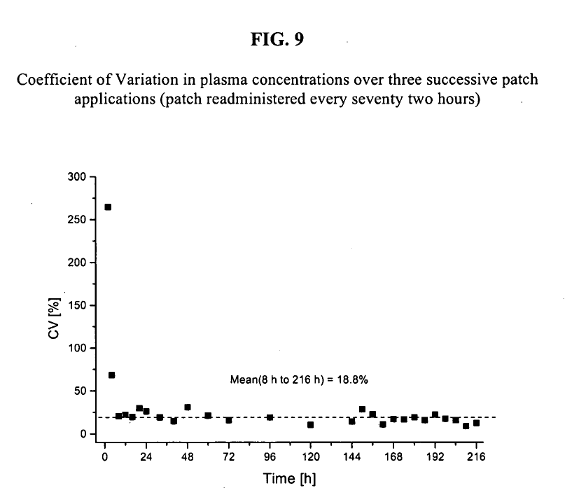

An analysis was made of the pharmacokinetics of two adhesive matrix-type sufentanil patches developed by Durect Corporation, as reported in WO 2006/047362. Each of the patches had an application area of 2.0 cm². Each of the matrices had an identical composition and sufentanil concentration. The first patch was a “thin” patch containing 0.91 mg. of sufentanil base. The second patch was a “thick” patch containing 1.7 mg. of sufentanil base. Plasma levels over 160 hours of patch application are reproduced in FIG. 6 hereto, and compared to the pharmacokinetics of the patch of Example 3. FIG. 7 plots normalized plasma levels over time, and again gives a comparison to the patch of Example 3.

### Example 7

### Solubility Recrystallization Study

The recrystallization study for determination of drug solubility in polymer was carried out by manufacturing patches having different concentrations of drug in each polymer. After manufacture the resulting laminate was free of undissolved drug. Patches were obtained from the laminate by dye cutting. The single patches were stored in four-side-sealing-pouches of composite material absolutely tight against light and humidity and stored at 25° C./60% RH or unsealed in a climate chamber of 40° C./75% RH. The patches were analysed visually and macroscopically for crystals after 1, 2 and 4 weeks of storage. The time, the amount and the size of crystals was assessed. Concentrations yielding crystals after 4 weeks under stress of 40° C./75% RH were judged above solubility; the next lower concentration revealing no crystals after 4 weeks under stress of 40° C./75% RH was judged below solubility. Solubility was determined to be in between those two concentrations of drug loading. Results are reported in Table F.

TABLE E

| | | | Solubility assessed by |
|---|---|---|---|
| | Name polymer | Polymer type | recrystallization study |
| 1 | Durotak ™ 387-2516 | Polacrylate | 7 ± 1.5% |
| | | copolymer | |
| 2 | Bio-PSA ™ 7-4602 | Silicone | 7 ± 1.5% |
| | | elastomer | |
| 3 | Oppanol ™ B100 and | Polyisobutylene | 2.5 ± 0.3% |
| | B10 | | |

### Example 8

### Multiple Dose Bioavailability Testing of Sufentanil Patch

A single center, three-dose, open label study to investigate the safety, tolerability and pharmacokinetics of sufentanil transdermal patches replaced every seventy two hours was undertaken using a parallel groups design. FIG. 8 is a graphical depiction of in vivo plasma levels over time for a 2.5 cm² patch constructed according to the present invention containing 0.625 mg of sufentanil base, in a phase I multi-dose bioavailability study in healthy volunteers (n=7) over seven days. Other kinetic variables are reported below in Tables F and G.

TABLE F
SUMMARY CONCENTRATIONS [PG/ML]

| Subj. | 0 | 2 | 4 | 8 | 12 | 16 | 20 | 24 |
|---|---|---|---|---|---|---|---|---|
| N | 7 | 7 | 7 | 7 | 7 | 7 | 7 | 7 |
| Mean | 0.00 | 0.30 | 7.91 | 31.39 | 41.31 | 46.78 | 44.23 | 50.09 |
| SD | 0.00 | 0.79 | 5.41 | 6.46 | 9.22 | 9.12 | 13.18 | 13.08 |
| SE | 0.00 | 0.30 | 2.04 | 2.44 | 3.49 | 3.45 | 4.98 | 4.94 |
| CV | — | 264.6 | 68.4 | 20.6 | 22.3 | 19.5 | 29.8 | 26.1 |
| Min | 0.00 | 0.00 | 2.59 | 21.76 | 26.01 | 36.49 | 28.73 | 40.11 |
| Q1 | 0.00 | 0.00 | 5.21 | 27.71 | 36.28 | 37.23 | 31.00 | 40.78 |
| Med | 0.00 | 0.00 | 5.51 | 30.12 | 40.62 | 50.89 | 47.06 | 43.13 |
| Q3 | 0.00 | 0.00 | 9.00 | 35.48 | 47.39 | 54.69 | 54.11 | 64.08 |
| Max | 0.00 | 2.08 | 19.10 | 41.78 | 54.77 | 56.59 | 63.67 | 72.92 |
| GeoM | — | 2.08 | 6.66 | 30.81 | 40.36 | 45.99 | 42.52 | 48.79 |
| G_CV | — | — | 68.7 | 21.2 | 24.3 | 20.3 | 31.4 | 24.4 |
| N\|x > 0 | 0 | 1 | 7 | 7 | 7 | 7 | 7 | 7 |
| Subj. | 32 | 40 | 48 | 60 | 72 | 96 | 120 | 144 |
| N | 7 | 7 | 7 | 7 | 7 | 7 | 7 | 7 |
| Mean | 44.14 | 38.70 | 42.54 | 32.74 | 30.10 | 63.96 | 47.37 | 32.73 |
| SD | 8.42 | 5.70 | 13.16 | 6.94 | 4.74 | 12.17 | 4.95 | 4.64 |
| SE | 3.18 | 2.16 | 4.97 | 2.62 | 1.79 | 4.60 | 1.87 | 1.75 |
| CV | 19.1 | 14.7 | 30.9 | 21.2 | 15.8 | 19.0 | 10.4 | 14.2 |
| Min | 35.02 | 31.96 | 29.74 | 26.53 | 25.90 | 48.92 | 37.50 | 25.48 |
| Q1 | 36.21 | 33.54 | 34.82 | 27.97 | 26.44 | 58.26 | 45.26 | 28.80 |
| Med | 45.03 | 39.45 | 35.98 | 29.90 | 27.87 | 62.04 | 48.53 | 32.28 |
| Q3 | 45.73 | 44.63 | 53.14 | 39.16 | 35.75 | 65.88 | 50.48 | 37.91 |
| Max | 60.40 | 45.72 | 67.32 | 45.44 | 37.44 | 88.58 | 52.42 | 38.38 |
| GeoM | 43.50 | 38.34 | 41.03 | 32.17 | 29.79 | 63.05 | 47.12 | 32.44 |
| G_CV | 18.3 | 14.9 | 28.8 | 19.9 | 15.3 | 18.0 | 11.2 | 14.7 |
| N\|x > 0 | 7 | 7 | 7 | 7 | 7 | 7 | 7 | 7 |
| Subj. | 150 | 156 | 162 | 168 | 174 | 180 | 186 | 192 |
| N | 7 | 7 | 7 | 7 | 7 | 7 | 7 | 7 |
| Mean | 58.08 | 77.68 | 65.51 | 69.22 | 62.36 | 54.91 | 52.34 | 50.73 |
| SD | 16.57 | 17.59 | 7.14 | 11.67 | 10.44 | 10.47 | 8.33 | 11.25 |
| SE | 6.26 | 6.65 | 2.70 | 4.41 | 3.94 | 3.96 | 3.15 | 4.25 |
| CV | 28.5 | 22.6 | 10.9 | 16.9 | 16.7 | 19.1 | 15.9 | 22.2 |
| Min | 40.08 | 61.79 | 58.27 | 54.57 | 47.16 | 41.81 | 43.69 | 40.55 |
| Q1 | 40.48 | 65.93 | 62.10 | 61.96 | 54.14 | 48.83 | 46.73 | 41.46 |
| Med | 54.93 | 75.32 | 62.35 | 65.15 | 61.88 | 49.83 | 49.98 | 48.00 |
| Q3 | 78.43 | 77.99 | 66.78 | 79.36 | 71.75 | 65.89 | 60.93 | 63.57 |
| Max | 78.70 | 115.39 | 80.29 | 89.44 | 75.80 | 71.45 | 66.66 | 69.29 |
| GeoM | 56.07 | 76.25 | 65.20 | 68.42 | 61.59 | 54.08 | 51.80 | 49.75 |
| G_CV | 29.4 | 20.3 | 10.3 | 16.5 | 17.3 | 18.8 | 15.3 | 21.2 |
| N\|x > 0 | 7 | 7 | 7 | 7 | 7 | 7 | 7 | 7 |
| Subj. | 198 | 204 | 210 | 216 | 222 | 228 | 240 | 264 |
| N | 7 | 7 | 7 | 7 | 7 | 7 | 7 | 7 |
| Mean | 46.63 | 39.40 | 35.26 | 34.10 | 22.74 | 12.75 | 8.61 | 4.96 |
| SD | 8.15 | 6.21 | 3.09 | 4.18 | 2.94 | 2.02 | 1.58 | 1.48 |
| SE | 3.08 | 2.35 | 1.17 | 1.58 | 1.11 | 0.76 | 0.60 | 0.56 |
| CV | 17.5 | 15.8 | 8.8 | 12.3 | 12.9 | 15.8 | 18.4 | 29.9 |
| Min | 38.32 | 30.17 | 31.26 | 28.26 | 18.36 | 10.26 | 6.36 | 2.93 |
| Q1 | 38.81 | 35.59 | 33.03 | 29.86 | 20.89 | 10.54 | 7.01 | 4.02 |
| Subj. | 0 | 2 | 4 | 8 | 12 | 16 | 20 | 24 |
| Med | 44.51 | 38.46 | 34.80 | 34.16 | 22.51 | 12.27 | 8.85 | 4.72 |
| Q3 | 53.93 | 44.68 | 38.20 | 39.01 | 25.36 | 14.27 | 10.35 | 6.21 |
| Max | 60.35 | 49.05 | 40.39 | 39.18 | 27.16 | 15.62 | 10.56 | 7.46 |
| GeoM | 46.05 | 38.98 | 35.14 | 33.88 | 22.58 | 12.62 | 8.48 | 4.77 |
| G_CV | 17.1 | 16.0 | 8.6 | 12.5 | 13.1 | 16.0 | 19.1 | 30.7 |
| N\|x > 0 | 7 | 7 | 7 | 7 | 7 | 7 | 7 | 7 |

TABLE G
PHARMACOKINETIC VARIABLES

| | AUC_{0-72sd} | AUC_{0-72,md} | AUC_{0-72,md}/ | C_{max0-72,sd} | C_{max0-72,md} | C_{max0-72,md}/ | t_{max0-72,md} | t_{max0-72,md} | Cₐᵥ | Cₘᵢₙ | |
|---|---|---|---|---|---|---|---|---|---|---|---|
| SUBJ | [pg/ml * h] | [pg/ml * h] | AUC_{0-72sd} | [pg/ml] | [pg/ml] | C_{max0-72,sd} | [h] | [h] | [pg/ml] | [pg/ml] | PTF |
| 001 | 2218.3 | 3379.7 | 1.52 | 41.003 | 71.870 | 1.75 | 32.00 | 156.00 | 46.94 | 25.48 | 0.99 |
| 002 | 2350.2 | 4275.8 | 1.82 | 50.844 | 115.394 | 2.27 | 20.00 | 156.00 | 59.39 | 29.86 | 1.44 |
| 003 | 2279.6 | 3978.9 | 1.75 | 45.731 | 78.699 | 1.72 | 32.07 | 150.00 | 55.26 | 34.25 | 0.80 |
| 004 | 3391.7 | 4041.1 | 1.19 | 72.922 | 71.745 | 0.98 | 24.00 | 174.00 | 56.13 | 34.12 | 0.67 |
| 005 | 3181.8 | 4175.5 | 1.31 | 64.077 | 79.359 | 1.24 | 24.00 | 168.00 | 57.99 | 32.13 | 0.81 |
| 006 | 2514.2 | 3862.1 | 1.54 | 63.665 | 89.439 | 1.40 | 20.03 | 168.00 | 53.64 | 28.80 | 1.13 |
| 008 | 2538.0 | 3403.9 | 1.34 | 50.886 | 62.169 | 1.22 | 16.00 | 162.00 | 47.28 | 32.28 | 0.63 |
| N | 7 | 7 | 7 | 7 | 7 | 7 | 7 | 7 | 7 | 7 | 7 |
| MEAN | 2639.1 | 3873.8 | 1.50 | 55.590 | 81.239 | 1.51 | 24.01 | 162.00 | 53.80 | 30.99 | 0.93 |
| STD | 461.2 | 355.1 | 0.23 | 11.491 | 17.250 | 0.43 | 6.12 | 8.49 | 4.93 | 3.16 | 0.29 |
| SE | 174.3 | 134.2 | 0.09 | 4.343 | 6.520 | 0.16 | 2.31 | 3.21 | 1.86 | 1.19 | 0.11 |
| CV | 17.5 | 9.2 | 15.43 | 20.6 | 21.2 | 28.62 | 25.49 | 5.24 | 9.17 | 10.18 | 30.84 |
| MIN | 2218.3 | 3379.7 | 1.19 | 41.003 | 62.169 | 0.98 | 16.00 | 150.00 | 46.94 | 25.48 | 0.63 |
| Q1 | 2279.6 | 3403.9 | 1.31 | 45.731 | 71.745 | 1.22 | 20.00 | 156.00 | 47.28 | 28.80 | 0.67 |
| MED | 2514.2 | 3978.9 | 1.52 | 50.886 | 78.699 | 1.40 | 24.00 | 162.00 | 55.26 | 32.13 | 0.81 |
| Q3 | 3181.8 | 4175.5 | 1.75 | 64.077 | 89.439 | 1.75 | 32.00 | 168.00 | 57.99 | 34.12 | 1.13 |
| MAX | 3391.7 | 4275.8 | 1.82 | 72.922 | 115.394 | 2.27 | 32.07 | 174.00 | 59.39 | 34.25 | 1.44 |
| GeoM | 2607.0 | 3859.4 | 1.48 | 54.582 | 79.831 | 1.46 | 23.35 | | 53.60 | 30.84 | 0.89 |
| Geo_CV | 16.7 | 9.4 | 15.5 | 20.9 | 19.9 | 28.6 | 26.0 | | 9.4 | 10.6 | 29.9 |

It is important to add that the average amount of sufentanil delivered from each patch was 0.34 mg, which translates to a loss of about 54% for each patch. It is also important to note that plasma concentrations from the 2.5 cm² patch, once the minimum effective plasma concentration is reached (30 pg/ml), never fall below the 30 pg/ml minimum effective plasma concentration as long as the patch is re-administered every three days.

The average plasma concentrations during the third patch administration, after steady state had been reached, was 53.8 pg/mL. Based on the osmotic pump study reported in Fisher et al. (2003), Anesthesiology 99(4): 929-37, it can be assumed that a steady state delivery of 1 mcg/hr sufentanil leads to an average plasma concentration of about 15.2 pg/ml. Based on the 53.8 pg/mL average plasma concentration observed in this study, it appears that the 2.5 cm² patch of the present invention achieves a steady state delivery rate of about 3.5 mcg/hr.

### Example 9

### Calculation of Transdermal Delivery Rate to Achieve Target Sufentanil Levels

A calculation was performed to determine the theoretical transdermal delivery rate of sufentanil needed to achieve various target sufentanil plasma levels, starting with a minimum patch size of 5 cm². Based on the 0.104 mcg/kg/h average intravenous infusion rate reported in the literature for achieving sustained analgesia, and the 60 pg/ml plasma concentration required for sustained analgesia, it was determined that the delivery rate needed to achieve the minimum level of effective analgesia (i.e. 30 pg/ml), was 0.050 mcg/kg/h (assuming 100% bioavailability from the patch), which equates to 3.5 mcg/h for a 70 kg. adult. If the minimum patch size is 5 cm², Table H would present the target transdermal delivery rates and sufentanil plasma levels.

TABLE H

| | | Target Sufentanil |
|---|---|---|
| Patch size | Transdermal delivery rate | Plasma level |
| 5 cm² | 3.5 microg/h | 30 pg/ml |
| | | (min., lower back pain) |
| 10 cm² | 7.0 microg/h | 60 pg/ml |
| | | (average) |
| 15 cm² | 10.5 microg/h | 90 pg/ml |
| | | (terminal pain) |
| 20 cm² | 14.0 microg/h | 120 pg/ml |
| | | (tolerance) |

Contrary to these expectations, the delivery of 3.5 mcg/hr by the patches of the present invention is actually able to achieve a steady state plasma concentration of 53.8 pg/ml.

### Example 10

### Comparative Testing of Saturated Sufentanil Patch with Irritation Reducing Agents

Patches comprising 2.94% (w/w) sufentanil were prepared using a PIB matrix material. It had previously been determined that sufentanil had a saturation solubility within this patch material of about 2.5% (w/w). Thus, the amount of sufentanil in the patches exceeded the saturation solubility of the patches.

In a first group of patches, no irritation reducing agents were included. In a second group of patches, very small particles of CGP were included throughout the matrix.

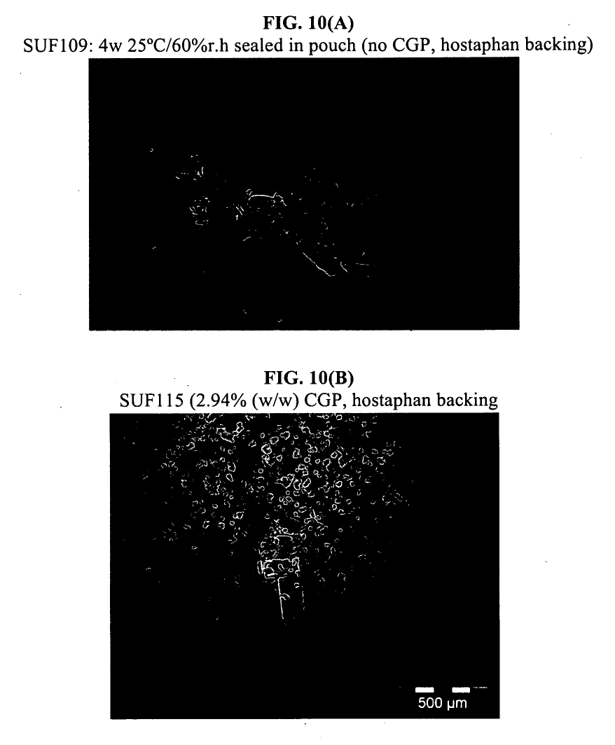

As seen in FIG. 10(A), the patches without irritation agents were found to have sufentanil crystals occurring as localized agglomerates of a relatively large size. However, as seen in FIG. 10(b), the patches with CGP were found to have a sufentanil crystals occurring homogenously throughout the entire matrix, and found not to have sufentanil crystals occurring as localized agglomerates.

### Example 11

### Sufentanil Crystallization in Patches with Backing that Including Comb Structures

Patches with a non-woven Corovin backing with a comb structure comprising 2.94% (w/w) sufentanil were prepared using a PIB matrix material with CGP particles. It had previously been determined that sufentanil had a saturation solubility within this patch material of about 2.5% (w/w). Thus, the amount of sufentanil in the patches exceeded the saturation solubility of the patches.

As seen in FIG. 11, the matrix (pictured on the left) contained CGP particles and no sufentanil crystals, and the corners of the combs (pictured on the right) contained sufentanil crystals. Sufentanil crystals were not observed in other areas of the patch.

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1) A method of treatment comprising:
a) adhering a first sufentanil patch system having a first sufentanil load to a first location on the skin of a patient in need of treatment for pain, for a first period of at least three days; and
b) delivering sufentanil from said first sufentanil patch system to said patient at a defined rate in which either
(1) said first patch system demonstrates a normalized Cₘₐₓ of greater than 12 ng/(ml·(mg/hr)),
(2) said first patch system achieves a mean plasma concentration divided by said defined rate of greater than 1.4*10⁻⁵ hr/ml,
(3) said first patch system demonstrates an 80% Tₘₐₓ of 18 hours or less,
(4) said first patch system demonstrates a load relative Cₘₐₓ of 0.12 ng/ml·mg or greater, or combinations thereof.

2) The method of claim 1, wherein said first period is three days.

3) The method of claim 1, wherein said defined rate constitutes the steady state rate of flux when said patch system is sequentially removed and replaced by successive patch systems each having said first sufentanil load.

4) The method of claim 1, wherein said first sufentanil patch system achieves a mean plasma concentration divided by nominal flux of greater than 1.0*10⁻⁵ hr/ml.

5) The method of claim 1, wherein said first sufentanil patch system is the first in sequence patch system, said patient has an existing opioid demand equipotent to n·90 mg/d of oral morphine, and said first patch system delivers n·(3.5±0.5) mcg/hr of sufentanil, wherein n is an integer of from 1 to 5.

6) The method of claim 1, wherein said first patch system demonstrates a normalized Cₘₐₓ of greater than 14, 16 or 18 ng/(ml·(mg/hr)).

7) The method of claim 1, wherein said first patch system demonstrates an 80% Tₘₐₓ of 16, 14 or 12 hours or less.

8) The method of claim 1, wherein said first patch system demonstrates a Tₘₐₓ of 24, 22, 20, or 18 hours or less.

9) The method of claim 1, wherein said first patch system comprises a 50% Tₘₐₓ of less than 10 hours, and a t_{1/2} after patch removal of less than 10 hours.

10) The method of claim 1, wherein said first patch system demonstrates a load relative Cₘₐₓ of 0.14, 0.16, 0.18, or 0.20 ng/ml·mg or greater.

11) The method of claim 1, wherein said first patch system, after reaching Cₘₐₓ and before the end of said first period, produces sufentanil plasma concentrations of 65%, 60%, 55% or 50% less than said Cₘₐₓ.

12) The method of claim 1, wherein said first patch system delivers 65, 70 or 75 wt. % or greater of said initial sufentanil loading to said patient during said first period.

13) The method of claim 1, wherein said first patch system demonstrates a standardized Cₘₐₓ of 0.01, 0.02, 0.03 ng/ml·cm² or greater.

14) The method of claim 1, wherein said first patch system has an in vivo flux rate of n·(3.5±0.5) mcg/hr, wherein n is 1, 2, 3, 4 or 5.

15) The method of claim 1, wherein said first patch system has a standardized in vivo flux rate of 2.0±0.5 mcg/hr·cm².

16) The method of claim 1, wherein said pain is persistent moderate to severe chronic pain.

17) The method of claim 1, wherein said pain is acute.

18) The method of claim 1, wherein said patient is opioid tolerant.

19) The method of claim 1, wherein said sufentanil is present in said first patch system in a concentration of from about 0.1 to about 0.5 mg/cm².

20) The method of claim 1, wherein said first patch system comprises a matrix, and said matrix comprises from about 2 to about 4 wt. % sufentanil base.

21) The method of claim 1, wherein said first patch system comprises a matrix, and said sufentanil is present below its solubility limit in said matrix.

22) The method of claim 1, wherein said first period is said initial period, said patient experiences inadequate pain control during said initial period, further comprising adhering a second sufentanil patch system to the skin of said patient after said first period, for a second period of at least three days, wherein said first and second patch systems are defined by an identical composition and size.

23) The method of claim 1, wherein said first period is said initial period, said patient experiences inadequate pain control during said initial period, further comprising adhering a second sufentanil patch system to the skin of said patient after said first period, for a second period of at least three days, wherein said first and second patch systems are defined by an identical composition and a different size.

24) The method of claim 1, wherein said first sufentanil patch system demonstrates a Cₘₐₓ having a coefficient of variation of 50%, 40%, 30%, 20% or less.

25) The method of claim 1, wherein said first sufentanil patch system comprises one or more sufentanil patches having a surface area of 1.75, 3.5, 5.25, 7.0 or 8.75.

26) The method of claim 1, wherein said sufentanil patch system comprises calcium glycerophosphate.

27) A transdermal patch comprising sufentanil or an analog thereof, wherein:
a) said patch comprises a protective flexible cover, an intermediate active layer having an adhesive surface opposite said protective cover, and a removable cover adjacent said adhesive surface, and
b) said patch system either
(1) demonstrates a normalized Cₘₐₓ of greater than 12 ng/(ml·(mg/hr)),
(2) achieves a mean steady state plasma concentration and a mean steady state in vivo flux, said mean steady state plasma concentration divided by said mean steady state in vivo flux is greater than 1.4*10⁻⁵ hr/ml,
(3) demonstrates an 80% Tₘₐₓ of 18 hours or less,
(4) demonstrates a load relative Cₘₐₓ of 0.12 ng/ml·mg or greater, or
(5) combinations thereof.

28) A transdermal patch comprising:
a) a protective flexible cover,
b) an intermediate active layer comprising:
i) sufentanil or an analog thereof;
ii) a non-saccharide polyhydric alcohol phosphate ester, or a pharmaceutically acceptable salt thereof; and
iii) an adhesive surface opposite said protective cover, and
c) a removable cover adjacent said adhesive surface.

29) The transdermal patch of claim 28, wherein said non-saccharide polyhydric alcohol phosphate ester is calcium glycerophosphate.

30) A transdermal patch comprising:
a) a protective flexible cover,
b) an intermediate active layer comprising:
i) sufentanil or an analog thereof;
ii) greater than 50 wt. % polyisobutylene; and
iii) an adhesive surface opposite said protective cover, and
c) a removable cover adjacent said adhesive surface.

31) The transdermal patch of claim 30 wherein said polyisobutylene comprises:
a) low molecular weight polyisobutylene, having a molecular weight less than 100,000 g/mol; and
b) high molecular weight polyisobutylene, having a molecular weight of greater than 650,000 g/mol.

32) The transdermal patch of claim 30, wherein said intermediate layer further comprises polybutene, wherein the weight ratio of polyisobutylene to polybutene is from about 2:1 to about 5:1.

33) A transdermal patch that is bioequivalent to a reference patch, wherein said reference patch is a matrix patch made by a process comprising:
a) dissolving in hexane 1.0 weight parts of a high molecular weight polybutene, 5.0 weight parts of a low molecular weight polyisobutylene, and 2.0 weight parts polybutene to form an adhesive mixture;
b) dissolving 0.25 weight parts of sufentanil base in ethyl acetate to form a drug mixture;
c) mixing 0.25 weight parts of calcium glycerophosphate in said drug mixture to form a CGP mixture;
d) mixing said adhesive mixture and said CGP mixture to form a mixed liquid, and stirring said mixed liquid for one hour;
e) coating said mixed liquid onto a release liner at a sufentanil concentration of about 0.25 mg/cm²;
f) drying said coated liner; and
g) applying a backing foil to said dried coated liner.
